(19) United States
(12) Patent Application Publication
(10) Pub. No.: US 2026/0151580 A1
(43) Pub. Date: Jun. 4, 2026

(54) Inner Membrane Tube Bronchial Catheter

(71) Applicant: Wuxi People's Hospital, Wuxi (CN)

(72) Inventors: Shibiao CHEN, Wuxi (CN); Zhiming ZHANG, Wuxi (CN); Jun ZHOU, Wuxi (CN); Zhentian XIONG, Wuxi (CN); Hong GAO, Wuxi (CN)

(73) Assignee: Wuxi People's Hospital, Wuxi (CN)

(21) Appl. No.: 19/110,075

(22) PCT Filed: Jun. 16, 2023
(86) PCT No.: PCT/CN2023/100699

§ 371 Date: Feb. 11, 2026

(30) Foreign Application Priority Data
Sep. 14, 2022 (CN) 202211112935.X

(51) Int. Cl. A61M 16/04; A61B 1/267

(52) U.S. Cl. CPC A61M 16/0434; A61B 1/2676; A61M 16/0463

(57) ABSTRACT

The present invention provides an inner membrane tube bronchial catheter. The head of the main ventilation tube is equipped with a main ventilation port and a lateral ventilation port, while the tail is provided with two ventilator interfaces. One of the two ventilation ports can be selectively connected to either ventilator interface, thereby forming a membrane ventilation lumen. A front sealing cuff and a rear sealing cuff are respectively arranged at the anterior and posterior sides of the lateral ventilation port, and they are each connected to an inflation tube and an inflation valve. During thoracic surgery, the front sealing cuff is inserted into one bronchial orifice, while the rear sealing cuff remains within the main airway, with the lateral ventilation port oriented toward the opposite bronchial orifice; by inflating or deflating the membranous ventilation tube, lung isolation can be easily achieved, thereby minimizing the catheter's outer diameter, reducing intubation-related injury, and providing a larger ventilation chamber with lower airway resistance when ventilating either lung. Moreover, suctioning and bronchoscopic examination are made more convenient. After the surgery, the front sealing cuff can be deflated and retracted into the main airway, thereby converting the device into a single-lumen endotracheal tube. The catheter has a simple design, is cost-effective, has a low complication rate, and holds significant potential for clinical application.

### FIELD OF INVENTION

The invention primarily relates to the field of medical ventilation catheters, and more particularly to an inner membrane tube bronchial catheter.

### BACKGROUND OF THE INVENTION

Lung isolation refers to an anesthesia technique that functionally or physically separates the ventilation pathways of both lungs at the tracheal carina or bronchial level. This technique has been revolutionary in thoracic anesthesia, as it provides an optimal surgical field for intrathoracic procedures, facilitating the rapid advancement of thoracic surgery, particularly thoracoscopic techniques.

Additionally, in emergency situations such as severe pulmonary hemorrhage, lung isolation is crucial for healthcare providers to perform rapid and effective interventions.

Currently, the primary objective of lung isolation is to induce lung collapse, thereby providing a broader surgical field to facilitate intrathoracic procedures. As a result, lung isolation is not only essential for pulmonary surgeries but is also frequently employed in other procedures involving thoracic organs. The absolute indications for lung isolation include:
(1) Preventing infectious secretions, blood, or clots from contaminating or obstructing the healthy-side bronchus and lung.
(2) Controlling ventilation distribution, such as in cases of bronchopleural fistula, bronchopleurocutaneous fistula, large unilateral cysts or bullae, tracheobronchial rupture, intraoperative opening of large airways, and life-threatening hypoxemia caused by unilateral lung disease.
(3) Unilateral bronchopulmonary lavage.
(4) Video-assisted thoracoscopic surgery (VATS)

Currently, the commonly used lung isolation methods in clinical practice include double-lumen endobronchial tubes, bronchial blockers, and single-lumen endotracheal tubes.

### (1) Double-Lumen Endobronchial Tube

The advantages of using a double-lumen endobronchial tube for lung isolation include the ability to facilitate suctioning and ventilation of both lungs, ease of performing bronchoscopy, and stable and effective lung isolation.

However, due to its relatively large outer diameter, it increases the risk of glottic injury during intubation. Additionally, compared to bronchial blockers, the ventilation lumens of a double-lumen endobronchial tube have smaller internal diameters, which can result in increased airway pressure during ventilation, potentially leading to barotrauma.

### (2) Bronchial Blocker

A bronchial blocker achieves lung isolation by occluding the bronchial opening on the surgical side, making it suitable for patients with narrower airways. However, its drawbacks include the difficulty in achieving adequate lung collapse, limited effectiveness in suctioning secretions, and challenges in lung recruitment after occlusion. Additionally, bronchial blocker placement requires precise positioning, making intubation more complex and limiting its widespread clinical adoption.

### (3) Single-Lumen Bronchial Tube

In patients shorter than 140 cm, neither a double-lumen endobronchial tube nor a bronchial blocker is feasible, leaving single-lumen endotracheal intubation as the only option. In this approach, the tube is positioned in the non-surgical lung for ventilation. However, the surgical-side lung remains non-ventilated, posing significant challenges for lung collapse, suctioning, and recruitment. As a result, this method is generally considered a last-resort alternative in clinical practice.

Although some alternative products have emerged in clinical practice, they still struggle to overcome the inherent limitations of the three basic structures mentioned above and fail to fully meet the clinical needs of anesthesiologists. Therefore, there is an urgent clinical need for a double-lumen endobronchial tube with a smaller outer diameter and a larger inner diameter, which facilitates lung collapse, suctioning, and lung recruitment while also being adaptable for use as a single-lumen endotracheal tube when necessary.

### SUMMARY OF THE INVENTION

To overcome the shortcomings of the prior art, the present invention provides an inner membrane tube bronchial catheter. The catheter comprises a main ventilation tube, the proximal end of which forms a main ventilation port; a front sealing cuff is provided on the outer wall of the main ventilation tube adjacent to the main ventilation port. An inflation tube and an inflation valve are provided to connect with the front sealing cuff. The tail of the main ventilation tube is respectively connected to a main ventilator interface and a lateral ventilator interface; on one side away from the main ventilation port and adjacent to the front sealing cuff, a lateral ventilation port is formed through the side wall of the main ventilation tube; on the side of the lateral ventilation port away from the front sealing cuff, a rear sealing cuff is provided on the outer wall of the main ventilation tube. An inflation tube and an inflation valve are provided to connect with the rear sealing cuff. One of the main ventilator interface or the lateral ventilator interface is connected to one of the main ventilation port or the lateral ventilation port, with a membrane ventilation lumen sealedly arranged at the connection site.

The tail end port of the main ventilation tube is connected to a main ventilator interface, and a lateral respiratory port is formed through the side wall of the main ventilation tube near the main ventilator interface. The lateral respiratory port is sealedly connected to a lateral ventilator interface.

The distance between the lateral ventilation port and the main ventilation port is 1 to 8 cm. At the corresponding position of the lateral ventilation port, the main ventilation tube is provided with a bend extending in the direction away from the lateral ventilation port, with an included angle between the central axes of the main ventilation tube on both sides of the bend ranging from 130° to 165°.

The lateral ventilation port is arranged on the wall of the main ventilation tube on the same side as the lateral ventilation port.

The two ends of the membrane ventilation lumen are respectively sealedly connected to the lateral ventilation port and the lateral respiratory port; or the two ends of the membrane ventilation lumen are respectively sealedly connected to the main ventilation port and the main ventilator interface.

The lateral respiratory port is arranged on the wall of the main ventilation tube on the side opposite to the lateral ventilation port.

The two ends of the membrane ventilation lumen are respectively sealedly connected to the lateral ventilation port and the main ventilator interface; or the two ends of the membrane ventilation lumen are respectively sealedly connected to the main ventilation port and the lateral respiratory port.

A video cable is embedded in the side wall of the main ventilation tube, with its video head at the proximal end positioned at the edge of the lateral ventilation port; the video cable passes through the wall at the tail of the main ventilation tube and is provided with a video interface, along with a matching display and power supply.

A suction chamber is embedded in the side wall of the main ventilation tube, with its opening positioned on the side adjacent to the front sealing cuff and away from the main ventilation port; at the tail of the main ventilation tube, the suction chamber penetrates the side wall and is provided with a suction tube and a negative-pressure connection port.

A portion of the side wall of the membrane ventilation lumen is bonded to the inner wall of the main ventilation tube. The bonding method includes, but is not limited to, thermal fusion, adhesive bonding, or ultrasonic welding, and the bonding sites include, but are not limited to, any of the following:

In one embodiment, the lateral respiratory port is arranged on the wall of the main ventilation tube on the same side as the lateral ventilation port. The two ends of the membrane ventilation lumen are respectively sealedly connected to the lateral ventilation port and the lateral respiratory port, and the outer wall of the membrane ventilation lumen on the side adjacent to the lateral ventilation port and the lateral respiratory port is bonded to the inner wall of the main ventilation tube on the same side as the lateral ventilation port and the lateral respiratory port.

Alternatively, the lateral respiratory port is arranged on the wall of the main ventilation tube on the same side as the lateral ventilation port. The two ends of the membrane ventilation lumen are respectively sealedly connected to the main ventilation port and the main ventilator interface; the outer wall of the membrane ventilation lumen on the side away from the lateral ventilation port and the lateral respiratory port is bonded to the inner wall of the main ventilation tube on the side opposite to the lateral ventilation port and the lateral respiratory port, and the outer wall of the membrane ventilation lumen is also bonded to the inner wall of the main ventilation tube between the lateral ventilation port and the main ventilation port.

Alternatively, the lateral respiratory port is arranged on the wall of the main ventilation tube on the side opposite to the lateral ventilation port. The two ends of the membrane ventilation lumen are respectively sealedly connected to the lateral ventilation port and the main ventilator interface, and the outer wall of the membrane ventilation lumen on the side adjacent to the lateral ventilation port is bonded to the inner wall of the main ventilation tube on the same side as the lateral ventilation port.

Alternatively, the lateral respiratory port is arranged on the wall of the main ventilation tube on the side opposite to the lateral ventilation port. The two ends of the membrane ventilation lumen are respectively sealedly connected to the main ventilation port and the lateral respiratory port, the outer wall of the membrane ventilation lumen on the side adjacent to the lateral respiratory port is bonded to the inner wall of the main ventilation tube on the same side as the lateral respiratory port, and the outer wall of the membrane ventilation lumen is also bonded to the inner wall of the main ventilation tube between the lateral ventilation port and the main ventilation port.

Advantages of the Invention: During tracheal intubation, the inserted catheter functions as a single-lumen endotracheal tube, with a smaller outer diameter compared to a double-lumen endobronchial tube, thereby effectively reducing the risk of intubation-related injury. During single-lung mechanical ventilation, the ventilation lumen provides a larger ventilation chamber than a conventional double-lumen endobronchial tube, improving ventilation efficiency. Additionally, intraoperative procedures such as bronchoscopy, lung collapse, suctioning, and lung recruitment can be performed with ease. Upon completion of single-lung ventilation, the catheter can be slightly withdrawn to convert it into a single-lumen endotracheal tube, minimizing airway irritation and eliminating the need for tube exchange in critically ill patients or those requiring ICU transfer.

### BRIEF DESCRIPTION OF DRAWINGS

FIG. 1 is a schematic diagram of the overall structure of the present invention.

FIG. 2 is an A-section schematic diagram of the main ventilation tube of the present invention.

FIG. 3 is a B-view longitudinal sectional schematic diagram of the main ventilation tube of the present invention.

FIG. 4 is a schematic diagram of the structure of the membrane ventilation lumen of the present invention.

FIG. 5 is an A-section schematic diagram of single-lung ventilation of the tube ventilation chamber according to the first embodiment of the present invention.

FIG. 6 is an A-section schematic diagram of single-lung ventilation of the membrane ventilation chamber according to the first embodiment of the present invention.

FIG. 7 is an A-section schematic diagram of single-lung ventilation of the membrane ventilation chamber according to the second embodiment of the present invention.

FIG. 8 is an A-section schematic diagram of single-lung ventilation of the tube ventilation chamber according to the second embodiment of the present invention.

FIG. 9 is an A-section schematic diagram of single-lung ventilation of the membrane ventilation chamber according to the third embodiment of the present invention.

FIG. 10 is an A-section schematic diagram of single-lung ventilation of the tube ventilation chamber according to the third embodiment of the present invention.

FIG. 11 is an A-section schematic diagram of single-lung ventilation of the tube ventilation chamber according to the fourth embodiment of the present invention.

FIG. 12 is an A-section schematic diagram of single-lung ventilation of the membrane ventilation chamber according to the fourth embodiment of the present invention.

FIG. 13 is a C cross-sectional schematic diagram of FIGS. 6, 7, 9, and 12 of the present invention.

FIG. 14 is a D cross-sectional schematic diagram of FIGS. 5, 8, 10, and 11 of the present invention.

FIG. 15 is an H cross-sectional schematic diagram of FIGS. 5 and 10 of the present invention.

FIG. 16 is an E cross-sectional schematic diagram of FIGS. 6 and 9 of the present invention.

FIG. 17 is a G cross-sectional schematic diagram of FIGS. 7 and 12 of the present invention.

FIG. 18 is an F cross-sectional schematic diagram of FIGS. 8 and 11 of the present invention.

FIG. 19 is a schematic diagram of the lateral connecting tube structure of the present invention.

In the FIG., 1, main ventilation tube; 10, tube ventilation chamber; 11, main ventilation port; 12, anterior sealing cuff; 13, main ventilator interface; 14, bend; 2, membrane ventilation lumen; 20, membrane ventilation chamber; 21, lateral ventilation port; 22, posterior sealing cuff; 23, lateral ventilator interface; 24, lateral breathing port; 3, lateral connecting tube; 31, first sealing tube; 32, second sealing tube; 33, third branch tube.

### DETAILED DESCRIPTION OF THE INVENTION

To facilitate a better understanding of the technical solutions of the present invention and to make its features, objectives, and advantages more apparent to those skilled in the art, the following detailed description is provided in conjunction with exemplary embodiments. It should be noted that these embodiments are presented solely for the purpose of illustrating the invention and should not be construed as limiting the scope of the claimed invention.

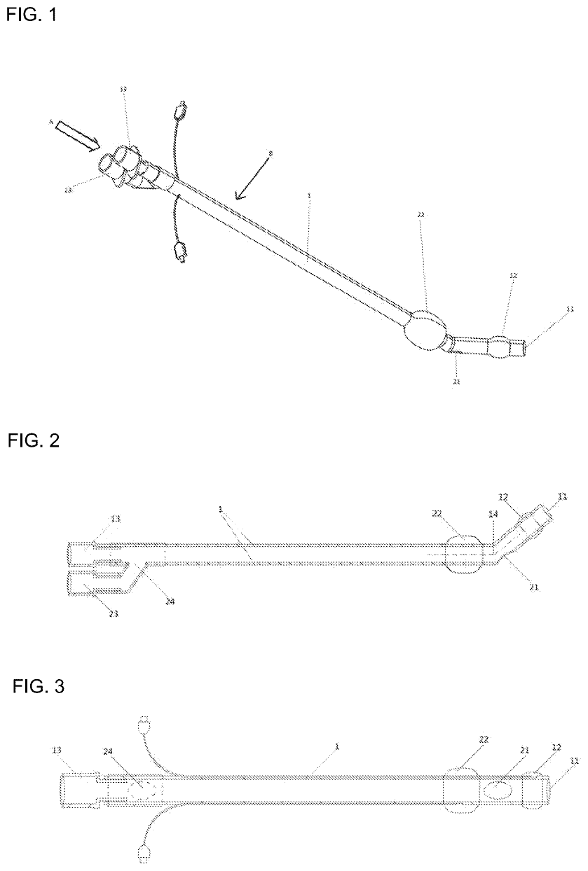

As shown in FIGS. 1 and 5 to 12, the inner membrane tube bronchial catheter of the present invention comprises a main ventilation tube (1), the proximal end port of which forms a main ventilation port (11). A front sealing cuff (12) is provided on the outer wall of the main ventilation tube (1) adjacent to the main ventilation port (11), with an inflation tube and an inflation valve connected to it. This constitutes the basic structure of a conventional single-lumen endotracheal tube; in use, the proximal end of the main ventilation tube (i.e., the main ventilation port (11) and the front sealing cuff (12)) is inserted into the patient's airway to an appropriate depth, and a suitable amount of gas is introduced into the front sealing cuff (12) via the inflation tube and inflation valve, after which the distal lumen of the main ventilation tube (1) is connected to the respiratory equipment to enable mechanical ventilation. In order to utilize the lumen of the main ventilation tube (1) to implement lung isolation technology and to achieve a maximal lumen cross-sectional area with a minimal outer diameter for the inserted tube body, as illustrated in FIGS. 2 and 3, the distal end of the main ventilation tube (1) is respectively connected to a main ventilator interface (13) and a lateral ventilator interface (23), and, on one side remote from the main ventilation port (11) and adjacent to the front sealing cuff (12), a lateral ventilation port (21) is formed through the side wall of the main ventilation tube (1). Consequently, the distal end of the main ventilation tube (1) is provided with two interfaces for connection to respiratory equipment—the main ventilator interface (13) and the lateral ventilator interface (23)—while the proximal end is equipped with two ventilation ports for communication with the left and right bronchi, namely the main ventilation port (11) and the lateral ventilation port (21).

By selecting either the main ventilator interface (13) or the lateral ventilator interface (23) and sealingly connecting it to either the main ventilation port (11) or the lateral ventilation port (21) with the membrane ventilation lumen (2) installed, the lumen of the main ventilation tube (1) is partitioned by the membrane ventilation lumen (2) into two isolated ventilation chambers: one being a tube ventilation chamber (10) formed by the exterior of the membrane ventilation lumen (2) and the lumen of the main ventilation tube (1) serving as the ventilation channel, and the other being a membrane ventilation chamber (20) formed by the interior of the membrane ventilation lumen (2) serving as the ventilation channel.

It should be noted that the diameter of the membrane ventilation lumen (2) must be matched to the inner dimensions of the main ventilation tube (1) so that, upon inflation, the membrane ventilation lumen (2) optimally fills the inner lumen of the main ventilation tube (1). If, when inflated, the diameter of the membrane ventilation lumen (2) is too large, the collapsed excess sidewall of the membrane ventilation lumen (2) will reduce the effective ventilatory lumen of the main ventilation tube (1); conversely, if it is too small, then when the membrane ventilation lumen (2) serves as the single-lung ventilation channel, the main ventilation tube (1) will not be fully utilized, thereby increasing airway resistance, which may adversely affect the patient's condition.

As shown, provided that the membrane ventilation lumen (2) is ensured not to rupture under video cable inspection, its membrane wall should be as thin as possible to minimize occupancy of the inner lumen of the main ventilation tube (1); simultaneously, the length of the membrane ventilation lumen (2) must match the distance between the two selected openings, since an overly long tube will inevitably twist and impair ventilation performance, while an excessively short tube will be disadvantageous for achieving a sealed bond at the connection openings at both ends. It is preferable to fabricate the membrane ventilation lumen (2) from soft, low-elasticity medical materials, such as PVC, silicone, or PE, and it is also preferable that the main ventilation tube (1) and the membrane ventilation lumen (2) be made of the same material to facilitate sealed bonding between them, which may be achieved using adhesive bonding, ultrasonic welding, thermal fusion, or other similar methods.

The membrane ventilation lumen 2 is sealingly connected to both the ventilator connection interface and the ventilation interface in four specific connection modes, as follows:
\textcircled{1} In the first mode, the two ends of the membrane ventilation lumen 2 are respectively connected to the main ventilation port 11 and the main ventilator interface 13.
\textcircled{2} In the second mode, the two ends of the membrane ventilation lumen 2 are respectively connected to the lateral ventilation port 21 and the side ventilator interface 23.
\textcircled{3} In the third mode, the two ends of the membrane ventilation lumen 2 are respectively connected to the main ventilation port 11 and the side ventilator interface 23.
\textcircled{4} In the fourth mode, the two ends of the membrane ventilation lumen 2 are respectively connected to the lateral ventilation port 21 and the main ventilator interface 13.

In a specific embodiment, the connection portions at both ends of the membrane ventilation lumen (2) are related to the positions and orientations of the main ventilator interface (13) and the lateral ventilator interface (23). To prevent the membrane ventilation chamber (20) from crossing into and obstructing the tube ventilation chamber (10), thereby impairing the smooth flow of ventilation, and to avoid any interference with the video cable during airway inspection, it is preferable that the terminal ends of the membrane ventilation lumen (2) be connected to the ventilator connection interfaces and ventilation ports located on the same side. The preferred scheme will be further described in detail below.

In order to achieve the objective of isolating ventilation to the left and right lungs via the tube ventilation chamber (10) and the membrane ventilation chamber (20) within the main ventilation tube (1), a rear sealing cuff (22) is provided on the outer wall of the main ventilation tube (1) on the side adjacent to the lateral ventilation port (21) and remote from the front sealing cuff (12). An inflation tube and an inflation valve are provided to connect with the rear sealing cuff (22). In use, the front sealing cuff (12) and the main ventilation port (11) at the proximal end of the main ventilation tube (1) are inserted into the left bronchus near the carina, such that the lateral ventilation port (21) located immediately behind the front sealing cuff (12) is positioned within the trachea, while the rear sealing cuff (22) is located posterior to the lateral ventilation port (21); upon inflation, the front sealing cuff (12) seals the left bronchial orifice, and the rear sealing cuff (22) seals the tracheal lumen posterior to the lateral ventilation port (21), thereby forming a passage connected to the left lung, namely “left bronchial passage—main ventilation port (11)—tube ventilation chamber (10) or membrane ventilation chamber (20)—main ventilator interface (13) or lateral ventilator interface (23)—respiratory equipment circuit,” while simultaneously forming a passage connected to the right lung, namely “right bronchial passage-lateral ventilation port (21)—membrane ventilation chamber (20) or tube ventilation chamber (10)—lateral ventilator interface (23) or main ventilator interface (13)—respiratory equipment circuit.” When the anesthetic machine is connected to the main ventilator interface (13) or lateral ventilator interface on the left lung passage while the interface on the right lung passage (either the lateral ventilator interface (23) or the main ventilator interface (13)) is left open to the atmosphere, left lung ventilation is implemented; conversely, when the anesthetic machine is connected to the interface at the right lung passage (either the lateral ventilator interface (23) or the main ventilator interface (13)) while the interface at the left lung passage (either the main ventilator interface (13) or the lateral ventilator interface (23)) is directly exposed, right lung ventilation is achieved. Under conditions of mechanical positive pressure ventilation, whether left or right single-lung ventilation is performed, the membrane ventilation lumen (2) will deform so that the ventilation chamber on the ventilated side opens while the chamber on the non-ventilated side remains closed, utilizing the sidewall of the main ventilation tube (1) as a constraint to maximize lumen utilization; specifically, if the tube ventilation chamber (10) is employed on the ventilated side, the membrane ventilation lumen (2) corresponding to the non-ventilated side's membrane ventilation chamber (20) will completely collapse, whereas if the membrane ventilation chamber (20) is used on the ventilated side, the corresponding membrane ventilation lumen (2) will fully inflate to occupy the entire lumen of the main ventilation tube (1), thereby occupying the tube ventilation chamber (10) on the non-ventilated side.

When performing suction or video scope examination on the non-ventilated side, the suction tube or video scope is introduced via the non-ventilated airway into the corresponding bronchus to perform the operation; when the suction tube or video scope traverses the region of the main ventilation tube (1) lumen occupied by the membrane ventilation lumen (2), it displaces the lateral wall of the membrane ventilation lumen (2) with minimal impact on single-lung ventilation; if lung recruitment is required, it is unnecessary to disconnect the respiratory circuit, as a simple breathing balloon may be directly connected to the non-ventilated side's main ventilator interface (13) or lateral ventilator interface (23) to perform recruitment, and once lung recruitment is complete, the breathing balloon may be detached to resume single-lung ventilation. If necessary, the suction tube may also be introduced into the bronchus on the non-ventilated side to perform suction, thereby accelerating the collapse of the non-ventilated lung.

Furthermore, as shown in FIGS. 1-2 and 5-12, the distance between the lateral ventilation port (21) and the main ventilation port (11) is 1 to 8 cm, a range adapted to the anatomical lengths of the patient's trachea and bronchi, ensuring that when the rear sealing cuff (22) is positioned within the main airway, the main ventilation port (11) and the front sealing cuff (12) at the proximal end of the main ventilation tube (1) can be accurately inserted into a unilateral bronchus without entering a subsequent bronchial bifurcation, thereby achieving optimal results; in adult product models, where patients have longer tracheas and bronchi, the distance between the lateral ventilation port (21) and the main ventilation port (11) is correspondingly longer, whereas in product models suitable for children of shorter stature, this distance is relatively shorter to match the length of the patient's trachea and bronchi.

For ease of positioning the main ventilation port (11) and front sealing cuff (12) of the main ventilation tube (1) into either the left or right bronchus, a bend (14) is provided at the location corresponding to the lateral ventilation port (21) in the main ventilation tube (1), with the included angle between the central axes on both sides of the bend (14) being 130° to 165°. A marking may be provided at the distal end of the main ventilation tube (1) in the direction of the main ventilation port (11) (i.e., anterior to the bend (14)) so that, during intubation, when the proximal end of the main ventilation tube (1) has entered the main airway, the operator may use this marking to orient the main ventilation port (11) toward the desired bronchial opening, thereby facilitating smooth insertion of the main ventilation port (11) and front sealing cuff (12) into the proximal portion of the target bronchus. Since the included angle of the central axes on both sides of the bend (14) is adapted to the bifurcation angles of the left and right bronchi relative to the trachea, proper placement reduces the tension imposed on the bronchial sidewall by the bending of the proximal end of the main ventilation tube (1), thereby lowering the risk of potential tissue damage. Moreover, the bend (14) is oriented opposite to the lateral ventilation port (21) and matched to the bifurcation structure of the main airway; thus, when the main ventilation port (11) and front sealing cuff (12) are inserted into one bronchial opening, the portion of the bend (14) oriented opposite to the lateral ventilation port (21) aligns with the opening of the opposite bronchus, thereby preventing any reduction in the effective ventilation area that might otherwise result from misalignment caused by twisting of the main ventilation tube (1).

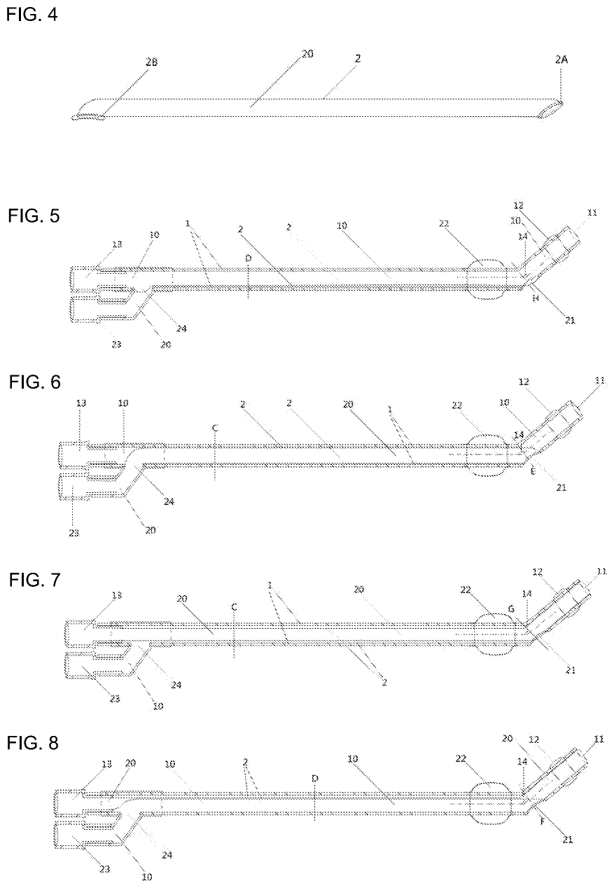

As shown in FIG. 4, the membrane ventilation lumen (2) is a tubular membrane structure with bonding portions 2A and 2B provided at its two ends to match the main ventilation tube (1); these bonding portions are designed to achieve an adapted bond with the main ventilation tube (1). In FIG. 2, the bonding portions 2A and 2B of the membrane ventilation lumen (2) correspond respectively to the lateral respiratory port (24) and the lateral ventilation port (21) as illustrated in FIGS. 2 and 3, and both are configured as rolled-over surface structures; if flat-interface bonding portions are employed, no special flip-over structure is required—only a certain surplus length needs to be reserved at bonding portions 2A and 2B to facilitate secure bonding, with any excess trimmed away after the bonding process is completed.

As shown in FIGS. 2-3, the distal end port of the main ventilation tube (1) is connected to the main ventilator interface (13), and a lateral respiratory port (24) is formed through the side wall of the main ventilation tube (1) in the vicinity of the main ventilator interface (13); after sealing, a lateral ventilator interface (23) is provided at the lateral respiratory port (24). This structural design is simple and cost-effective and can be implemented by creating a lateral cut hole at the distal end of the main ventilation tube (1). As shown in FIG. 19, one embodiment of the lateral ventilator interface (23) is achieved by employing an injection-molded, three-way tubular lateral connecting tube (3). The lateral connecting tube (3) is provided with a first sealing tube (31) and a second sealing tube (32) that are interconnected, and its external configuration is matched to both sides of the lateral respiratory port (24) of the main ventilation tube (1). By assembling and bonding the lateral connecting tube (3) to the outer walls on both sides of the lateral respiratory port (24), the end of the first sealing tube (31) is sealed with the outer wall of the main ventilation tube (1) at the head of the lateral respiratory port (24), and the end of the second sealing tube (32) is sealed with the outer wall of the main ventilation tube (1) at the tail of the lateral respiratory port (24), thereby allowing the third branch (33) of the lateral connecting tube (3) to communicate with the inner lumen of the main ventilation tube (1) via the lateral respiratory port (24), with a ventilator interface (23) correspondingly provided at the open end of the third branch (33). Of course, other embodiments for implementing the lateral ventilator interface (23) are possible and will not be described further herein.

The lateral respiratory port (24) could be positioned on the same side of the main ventilation tube (1) as the lateral ventilation port (21) or on the opposite side; however, it should not be positioned on any other side. Otherwise, regardless of how the membrane ventilation lumen (2) is bonded, its adhesion sites would not be aligned in the same direction. When the membrane ventilation lumen (2) collapses, the resulting membrane ventilation chamber (20) would undergo deformation, which would not only adversely affect the insertion of a suction tube or video scope through the membrane ventilation chamber (20) but also cause the collapsed membrane ventilation lumen (2) to form a buckled, sheet-like structure within the main ventilation tube (1). This would reduce the effective ventilation cross-sectional area of the tube ventilation chamber (10) and hinder the passage of a suction tube or video scope through the tube ventilation chamber (10), ultimately compromising mechanical ventilation and respiratory management.

As shown in FIGS. 2 and 5 to 8, the lateral respiratory port (24) is arranged on the wall of the main ventilation tube (1) on the same side as the lateral ventilation port (21). Accordingly, both ends of the membrane ventilation lumen (2) are preferably bonded to the interfacing portions on the same side of the main ventilation tube (1) to prevent any crossover between the membrane ventilation lumen (2) and the inner lumen of the main ventilation tube (1), thereby avoiding any intermingling of the tube ventilation chamber (10) and the membrane ventilation chamber (20) and preventing the collapsed membrane ventilation lumen (2) from obstructing ventilation when single-lung ventilation is implemented through the tube ventilation chamber (10). In this configuration, the membrane ventilation lumen (2) has two preferred bonding schemes.

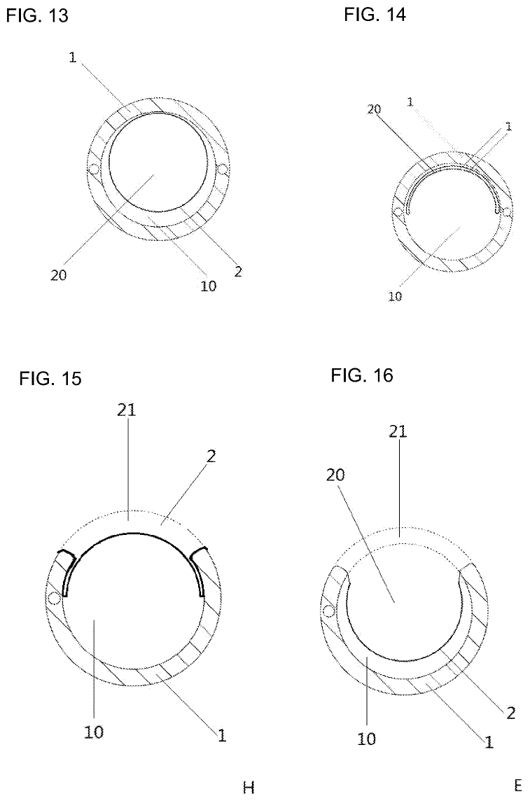

As shown in FIGS. 5-6, a first preferred scheme is provided wherein the two ends of the membrane ventilation lumen (2) are respectively sealedly connected to the lateral ventilation port (21) and the lateral respiratory port (24). In this configuration, the tube ventilation chamber (10) comprises the pathway defined by the main ventilation port (11), the portion of the inner lumen of the main ventilation tube (1) located outside the membrane ventilation lumen (2), and the main ventilator interface (13), while the membrane ventilation chamber (20) comprises the pathway defined by the lateral ventilation port (21), the inner lumen of the membrane ventilation lumen (2), and the lateral ventilator interface (23). In FIG. 5, a schematic illustration shows the operational state when single-lung ventilation is applied on the same side as the tube ventilation chamber (10):
under positive pressure within the tube ventilation chamber (10), the membrane ventilation lumen (2) is pressed and collapses onto the inner wall of the main ventilation tube (1) on the same side as the lateral ventilation port (21) and the lateral respiratory port (24), resulting in the segment of the membrane ventilation lumen (2) within the membrane ventilation chamber (20) being in a collapsed, closed state (as further illustrated in the cross-sectional view along line D in FIG. 14 and line H in FIG. 15). Conversely, FIG. 6 illustrates the state when single-lung ventilation is applied on the same side as the membrane ventilation chamber (20): under positive pressure within the membrane ventilation chamber (20), the membrane ventilation lumen (2) fully inflates, causing its sidewall to adhere tightly to the inner wall of the main ventilation tube (1), while the segment of the membrane ventilation lumen (2) within the tube ventilation chamber (10) is occupied by the inflated membrane ventilation lumen (2), forming an almost eliminated gap (as depicted in the cross-sectional views along line C in FIG. 13 and line E in FIG. 16).

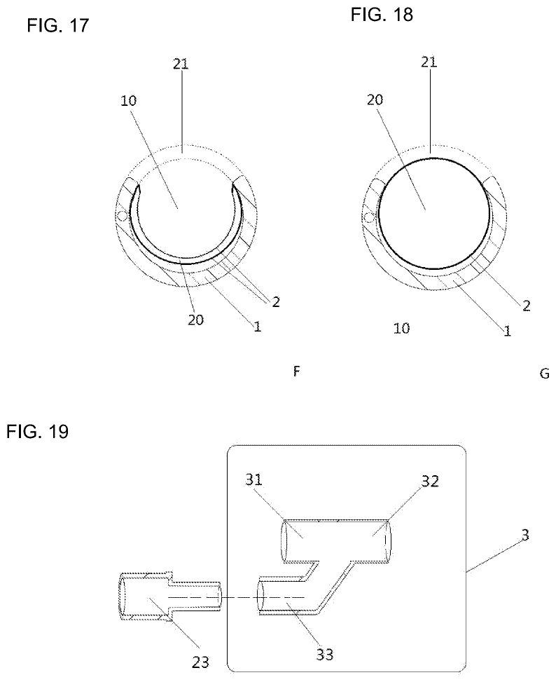

As shown in FIGS. 7-8, a second preferred scheme is provided wherein the two ends of the membrane ventilation lumen (2) are sealedly connected to the main ventilation port (11) and the main ventilator interface (13), respectively. In this configuration, the tube ventilation chamber (10) comprises the pathway “lateral ventilation port (21)—the inner lumen of the main ventilation tube (1) outside the membrane ventilation lumen (2)—lateral ventilator interface (23),” while the membrane ventilation chamber (20) comprises the pathway “main ventilation port (11)—the inner lumen of the membrane ventilation lumen (2)—main ventilator interface (13).” In the embodiment illustrated in FIG. 7, which represents the operational state when single-lung ventilation is applied on the same side as the membrane ventilation chamber (20), positive pressure within the membrane ventilation chamber (20) causes the membrane ventilation lumen (2) to fully inflate and its sidewall to closely adhere to the inner wall of the main ventilation tube (1), such that the segment of the membrane ventilation lumen (2) in the tube ventilation chamber (10) is occupied by the inflated membrane tube, nearly eliminating the gap (as illustrated in the cross-sectional views along line C in FIG. 13 and line G in FIG. 17). In contrast, the state shown in FIG. 8, representing single-lung ventilation on the same side as the tube ventilation chamber (10), demonstrates that under positive pressure within the tube ventilation chamber (10) the membrane ventilation lumen (2) is pressed and collapses against the inner wall of the main ventilation tube (1) on the side opposite the lateral ventilation port (21) and lateral respiratory port (24), causing the corresponding segment of the membrane ventilation lumen (2) in the membrane ventilation chamber (20) to collapse and close (as depicted in the cross-sectional views along line D in FIG. 14 and line F in FIG. 18).

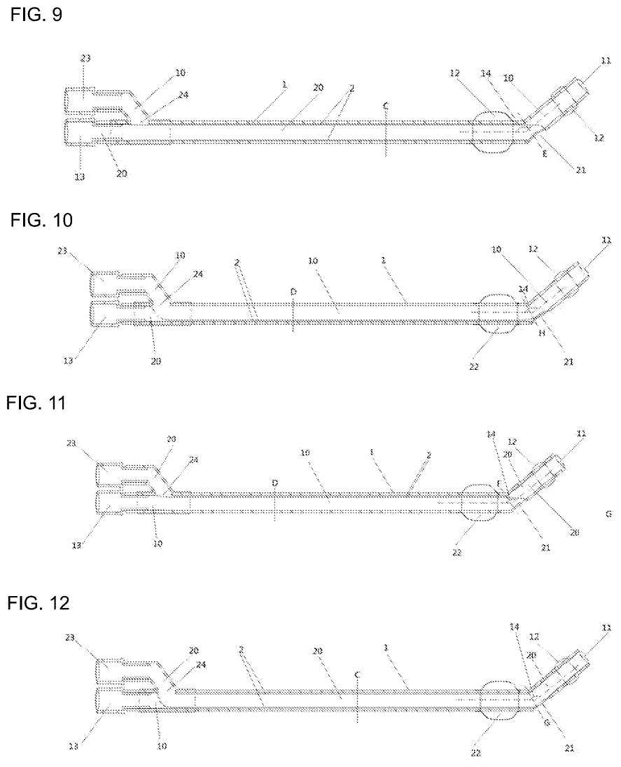

As shown in FIGS. 9-12, the lateral respiratory port (24) is arranged on the wall of the main ventilation tube (1) on the side opposite to the lateral ventilation port (21). In this configuration, the membrane ventilation lumen (2) may be bonded using one of two preferred schemes. In the first preferred scheme, as shown in FIGS. 9-10, the two ends of the membrane ventilation lumen (2) are respectively sealedly connected to the lateral ventilation port (21) and the main ventilator interface (13). In this arrangement, the tube ventilation chamber (10) comprises the pathway “main ventilation port (11)—the inner lumen of the main ventilation tube (1) outside the membrane ventilation lumen (2)—lateral ventilator interface (23),” while the membrane ventilation chamber (20) comprises the pathway “lateral ventilation port (21)—the inner lumen of the membrane ventilation lumen (2)—main ventilator interface (13).” In the embodiment illustrated in FIG. 9, which shows the operational state when single-lung ventilation is applied on the same side as the membrane ventilation chamber (20), positive pressure within the membrane ventilation chamber (20) causes the membrane ventilation lumen (2) to fully inflate, with its sidewall tightly adhering to the inner wall of the main ventilation tube (1), so that the segment of the membrane ventilation lumen (2) within the tube ventilation chamber (10) is occupied by the inflated membrane tube, nearly eliminating the gap (as illustrated in the cross-sectional views along line C in FIG. 13 and line E in FIG. 16). In contrast, FIG. 10 depicts the state when single-lung ventilation is applied on the same side as the tube ventilation chamber (10): under positive pressure within the tube ventilation chamber (10), the membrane ventilation lumen (2) is pressed and collapses against the inner wall of the main ventilation tube (1) on the same side as the lateral ventilation port (21) and the main ventilation port (13), resulting in the corresponding segment of the membrane ventilation lumen (2) in the membrane ventilation chamber (20) collapsing and closing (as shown in the cross-sectional views along line D in FIG. 14 and line H in FIG. 15).

As shown in FIGS. 11-12, a second preferred scheme is provided in which the two ends of the membrane ventilation lumen (2) are sealedly connected to the main ventilation port (11) and the lateral ventilator interface (23), respectively. In this configuration, the tube ventilation chamber (10) comprises the pathway “lateral ventilation port (21)—the inner lumen of the main ventilation tube (1) outside the membrane ventilation lumen (2)—main ventilator interface (13),” while the membrane ventilation chamber (20) comprises the pathway “main ventilation port (11)—the inner lumen of the membrane ventilation lumen (2)—lateral ventilator interface (23).” In the embodiment illustrated in FIG. 11, which represents the operational state when single-lung ventilation is applied on the same side as the tube ventilation chamber (10), positive pressure within the tube ventilation chamber (10) causes the membrane ventilation lumen (2) to be pressed and collapse against the inner wall of the main ventilation tube (1) on the same side as the main ventilation port (11) and the lateral ventilator interface (23), resulting in the segment of the membrane ventilation lumen (2) within the membrane ventilation chamber (20) collapsing and closing (as shown in the cross-sectional views along line D in FIG. 14 and line F in FIG. 18). Conversely, in the embodiment illustrated in FIG. 12, which represents the operational state when single-lung ventilation is applied on the same side as the membrane ventilation chamber (20), positive pressure within the membrane ventilation chamber (20) causes the membrane ventilation lumen (2) to fully inflate so that its sidewall closely adheres to the inner wall of the main ventilation tube (1), and the segment of the membrane ventilation lumen (2) within the tube ventilation chamber (10) is occupied by the inflated membrane tube, forming an almost eliminated gap (as depicted in the cross-sectional views along line C in FIG. 13 and line G in FIG. 17).

FIGS. 5-12 illustrate eight schematic representations of single-lung ventilation states corresponding to four optimal embodiments. In practice, both the front sealing cuff (12) and the rear sealing cuff (22) must be inflated with an appropriate amount of gas, with the front sealing cuff (12) positioned within the target bronchial lumen to block the annular gap between the inner wall of the target bronchus and the proximal lumen of the main ventilation tube (1), and the rear sealing cuff (22) positioned within the trachea to block the gap between the tracheal wall and the corresponding portion of the main ventilation tube (1). Accurate positioning is a prerequisite for successful lung isolation; misplacement—whether too deep or too shallow—will lead to failure of the lung isolation procedure. Positioning can be achieved via auscultation or direct visualization with a video scope, a clinical skill that anesthesiologists must master and which will not be further elaborated here. It is noteworthy that, in a minority of patients, variations in the right upper lobe bronchial opening may result in a higher or even carinal-level position, so that if the target bronchus for insertion of the main ventilation port (11) and front sealing cuff (12) is the right bronchus, the right upper lobe bronchus may be obstructed, preventing optimal lung collapse; since no clinical cases of an abnormally high left upper lobe bronchial opening have been reported, it is recommended that the target bronchus for insertion of the main ventilation port (11) and front sealing cuff (12) be the left bronchus in clinical applications.

At the conclusion of surgery and following the cessation of anesthesia, to reduce the pronounced circulatory fluctuations and coughing induced by compression of the carina by the proximal end of the main ventilation tube (1), the main ventilation tube (1) is retracted slightly before the end of the thoracic procedure so that the main ventilation port (11) and front sealing cuff (12) withdraw back into the trachea; meanwhile, the lateral ventilation port (21) and the rear sealing cuff (22) remain within the trachea. By selecting either the main ventilator interface (13) connected to the main ventilation port (11) or the lateral ventilator interface (23) and attaching the respiratory circuit thereto, dual-lung mechanical ventilation may be administered via the tube ventilation chamber (10) or the membrane ventilation chamber (20) that are connected to the main ventilation port (11). Specifically, in the embodiments illustrated in FIGS. 5-12, the specific states during single-lung ventilation, as shown in FIGS. 5, 7, 10, and 12, differ only in that the main ventilation port (11), front sealing cuff (12), lateral ventilation port (21), and rear sealing cuff (22) remain within the trachea rather than with the main ventilation port (11) and front sealing cuff (12) being positioned within the target bronchus during single-lung ventilation.

As shown in FIG. 4, the membrane ventilation lumen (2) is a tubular membrane structure with bonding portions 2A and 2B provided at both ends to match the main ventilation tube (1) to facilitate bonding. In FIG. 2, the bonding portions 2A and 2B of the membrane ventilation lumen (2) respectively correspond to the lateral respiratory port (24) and the lateral ventilation port (21) as illustrated in FIGS. 2 and 3, and adopt a rolled-over surface structure. If the bonding portions are designed as flat interfaces, no special flip-over structure is required; it is only necessary to reserve a sufficient length at the bonding portions 2A and 2B of the membrane ventilation lumen (2) to facilitate secure bonding, with any excess trimmed off once the bonding is completed.

Furthermore, to facilitate the positioning of the tube—especially for positioning the primary ventilation port (11) and the anterior sealing pouch (12) within the target bronchus, as well as for accurately aligning the lateral ventilation port (21) with the opening of the bronchus on the opposite side—a video cable may be embedded in the sidewall of the primary ventilation tube (1). The head of the video cable is arranged at the edge of the lateral ventilation port (21), thereby enabling simultaneous observation of the primary ventilation port (11), the anterior sealing pouch (12), and the bronchial openings on both sides. The corresponding video cable may be led out through a video interface from the sidewall at the distal end of the primary ventilation tube (1) and is provided with a matching display and power supply.

Furthermore, a suction chamber may be embedded in the sidewall of the main ventilation tube (1). The opening of this suction chamber is arranged on the side adjacent to the front sealing cuff (12) and away from the main ventilation port (11). A suction tube and a negative pressure connection port are led out from the sidewall at the distal end of the main ventilation tube (1). In this way, when the device is used as a single-lumen tube and dual-lung ventilation is maintained for an extended period, secretions entering the airway via the glottis and accumulating above the front sealing cuff (12) can be aspirated and cleared, thereby reducing the risk of respiratory infection.

Furthermore, to enhance the fixation of the membrane ventilation lumen (2) during single-lung ventilation and reduce the potential for relative slackness of the membrane ventilation lumen (2) caused by bending of the main ventilation tube (1)—thereby preventing the membrane ventilation lumen (2) from twisting and impeding smooth ventilation—a portion of the sidewall of the membrane ventilation lumen (2) may be bonded to the inner wall of the main ventilation tube (1). The bonding method may include, but is not limited to, thermal fusion, adhesive bonding, or ultrasonic welding, primarily to secure the collapsed portion of the membrane wall of the membrane ventilation lumen (2) that does not contribute to the beneficial effect, to the adjacent inner wall of the main ventilation tube (1); the bonding positions include, but are not limited to, any of the following:
As shown in FIGS. 5-6, the lateral respiratory port (24) is arranged on the wall of the main ventilation tube (1) on the same side as the lateral ventilation port (21), and the two ends of the membrane ventilation lumen (2) are respectively sealedly connected to the lateral ventilation port (21) and the lateral respiratory port (24). During transitions between left and right single-lung ventilation, the membrane wall of the membrane ventilation lumen (2) on the side away from the lateral ventilation port (21) and the lateral respiratory port (24) undergoes the primary deformation, thereby achieving collapse or inflation of the membrane ventilation lumen (2), while the outer wall of the membrane ventilation lumen (2) adjacent to these ports essentially remains unchanged. Based on this, the outer wall of the membrane ventilation lumen (2) adjacent to the lateral ventilation port (21) and the lateral respiratory port (24) may be bonded to the inner wall of the main ventilation tube (1) on the same side as the lateral ventilation port (21) and the lateral respiratory port (24). The bonding area is centered on the line connecting the lateral ventilation port (21) and the lateral respiratory port (24) and is preferably arranged symmetrically on both sides of this centerline, with the bonding width being less than half of the annular cross-sectional width of the membrane ventilation lumen (2), preferably between one-third and one-half, and the bonding length being limited by the distance between the adjacent lateral edges of the lateral ventilation port (21) and the lateral respiratory port (24).

As shown in FIGS. 7-8, the lateral respiratory port (24) is arranged on the wall of the main ventilation tube (1) on the same side as the lateral ventilation port (21), and the two ends of the membrane ventilation lumen (2) are sealedly connected to the main ventilation port (11) and the main ventilator interface (13), respectively. The outer wall of the membrane ventilation lumen (2) on the side away from the lateral ventilation port (21) and lateral respiratory port (24) may be bonded to the inner wall of the main ventilation tube (1) on the side opposite to the lateral ventilation port (21) and the lateral respiratory port (24), while in the region between the lateral ventilation port (21) and the main ventilation port (11), the outer wall of the membrane ventilation lumen (2) may also be bonded to the inner wall of the main ventilation tube (1). Bonding at these locations effectively eliminates any dead-end cavity between the lateral ventilation port (21) and the main ventilation port (11) along the outer wall of the membrane ventilation lumen (2), thereby preventing a suction tube or video scope from inadvertently entering this cavity during operation and making suctioning or video scope examinations easier.

As shown in FIGS. 9-10, the lateral respiratory port (24) is arranged on the wall of the main ventilation tube (1) on the side opposite to the lateral ventilation port (21); the two ends of the membrane ventilation lumen (2) are sealedly connected to the lateral ventilation port (21) and the main ventilator interface (13), respectively, and the outer wall of the membrane ventilation lumen (2) adjacent to the lateral ventilation port (21) is bonded to the inner wall of the main ventilation tube (1) on the same side as the lateral ventilation port (21). The operational principle is similar to that shown in FIGS. 5-6 and will not be repeated here.

As shown in FIGS. 11-12, the lateral respiratory port (24) is arranged on the wall of the main ventilation tube (1) on the side opposite to the lateral ventilation port (21), and the two ends of the membrane ventilation lumen (2) are sealedly connected to the main ventilation port (11) and the lateral respiratory port (24), respectively. The outer wall of the membrane ventilation lumen (2) adjacent to the lateral respiratory port (24) is bonded to the inner wall of the main ventilation tube (1) on the same side as the lateral respiratory port (24), and additionally, the outer wall of the membrane ventilation lumen (2) is also bonded to the inner wall of the main ventilation tube (1) in the region between the lateral ventilation port (21) and the main ventilation port (11). The underlying principle and method are similar to those illustrated in FIGS. 7-8 and will not be repeated here.

The foregoing embodiments are provided solely to illustrate the principles and advantages of the present patent application and are not intended to limit its scope. Those skilled in the art may modify or adapt these embodiments without departing from the spirit and scope of the present invention. Accordingly, all equivalent modifications or changes that may be made by persons of ordinary skill in the art, while remaining within the spirit and technical concepts disclosed herein, are intended to be encompassed by the claims of the present patent application.

We claim:

1. An inner membrane tube bronchial catheter characterized in that: it includes a main ventilation tube (1), the head end of the main ventilation tube (1) forms a main ventilation port (11), an anterior sealing sac (12) is provided on the outer wall of the main ventilation tube (1) near the main ventilation port (11), an inflation tube and an inflation valve are provided connected to the anterior sealing sac (12), the tail end of the main ventilation tube (1) is respectively connected to a main ventilator interface (13) and a side ventilator interface (23), a side ventilation port (21) is provided on the side of the main ventilation tube (1) away from the main ventilation port (11) near the anterior sealing sac (12), and a posterior sealing sac (22) is provided on the outer wall of the main ventilation tube (1) on the side of the main ventilation tube (1) away from the anterior sealing sac (12) near the side ventilation port (21), and the posterior sealing sac (22) is connected to the posterior sealing sac (22), an inflation tube and an inflation valve are provided; one of the main ventilator interface (13) and the side ventilator interface (23) is connected to one of the main airway (11) and the side airway (21) and a ventilation membrane tube (2) is provided in a sealed manner.

2. An inner membrane tube bronchial catheter according to claim 1, characterized in that: the end port of the main ventilation tube (1) is connected to a main ventilator interface (13), a side breathing port (24) is provided through the side wall of the main ventilation tube (1) near the main ventilator interface (13), and a side ventilator interface (23) is provided in a sealed connection to the side breathing port (24).

3. An inner membrane tube bronchial catheter according to claim 1, characterized in that: the distance between the side ventilation port (21) and the main ventilation port (11) is 1-8 cm; the main ventilation tube (1) has a bend (14) at the ventilation port (21) that is opposite to the direction of the side ventilation port (21), and the included angle between the central axes of the main ventilation tubes (1) on both sides of the bend (14) is 130°-165°.

4. An inner membrane tube bronchial catheter according to claim 2, characterized in that: the side breathing port (24) is disposed on the wall of the main ventilation tube (1) on the same side as the side ventilation port (21).

5. An inner membrane tube bronchial catheter according to claim 4, characterized in that: both ends of the ventilation membrane tube (2) are sealed and connected to the side ventilation port (21) and the side breathing port (24) respectively; or both ends of the ventilation membrane tube (2) are sealed and connected to the main ventilation port (11) and the main ventilator interface (13) respectively.

6. An inner membrane tube bronchial catheter according to claim 2, characterized in that: the side breathing port (24) is disposed on the wall of the main ventilation tube (1) opposite to the side ventilation port (21).

7. An inner membrane tube bronchial catheter according to claim 6, characterized in that: both ends of the ventilation membrane tube (2) are respectively sealed and connected to the side ventilation port (21) and the main ventilator interface (13); or both ends of the ventilation membrane tube (2) are respectively sealed and connected to the main ventilation port (11) and the side ventilation port (24).

8. An inner membrane tube bronchial catheter according to claim 1, characterized in that: a video cable is embedded in the side wall of the main ventilation tube (1), the video head at the head end of the video cable is set at the edge of the side ventilation port (21), and a video interface is set at the tail end of the main ventilation tube (1) on the side wall of the main ventilation tube (1), and a matching display screen and power supply are provided.

9. An inner membrane tube bronchial catheter according to claim 1, characterized in that: a suction cavity is embedded in the side wall of the main ventilation tube (1), the opening of the suction cavity is located on the side of the anterior sealing sac (12) away from the main ventilation port (11), and the suction cavity is provided with a suction tube and a negative pressure connection port at the end of the main ventilation tube (1) on the side wall of the main ventilation tube (1).

10. An inner membrane tube bronchial catheter according to claim 5 or 7, characterized in that: a portion of the sidewall of the ventilation membrane tube (2) is bonded to the inner wall of the main ventilation tube (1), the bonding method including but not limited to heat sealing, adhesive bonding or ultrasonic welding, and the bonding part including but not limited to one of the following: the side breathing port (24) is located on the wall of the main ventilation tube (1) on the same side as the side breathing port (21), both ends of the ventilation membrane tube (2) are respectively sealed and connected to the side breathing port (21) and the side breathing port (24), and the outer wall of the ventilation membrane tube (2) on the side adjacent to the side breathing port (21) and the side breathing port (24) is bonded to the inner wall of the main ventilation tube (1) on the same side as the side breathing port (21) and the side breathing port (24);
or alternatively, the side breathing port (24) is located on the wall of the main ventilation tube (1) on the same side as the side ventilation port (21). The two ends of the ventilation membrane tube (2) are sealed and connected to the main ventilation port (11) and the main ventilator interface (13) respectively. The outer wall of the ventilation membrane tube (2) away from the side ventilation port (21) and the side breathing port (24) is bonded to the inner wall of the main ventilation tube (1) on the opposite side of the side ventilation port (21) and the side breathing port (24). The outer wall of the ventilation membrane tube (2) is bonded to the inner wall of the main ventilation tube (1) between the side ventilation port (21) and the main ventilation port (11).
or alternatively, the side breathing port (24) may be located on the wall of the main ventilation tube (1) opposite to the side ventilation port (21). The two ends of the ventilation membrane tube (2) may be sealed and connected to the side ventilation port (21) and the main ventilator interface (13), respectively. The outer wall of the ventilation membrane tube (2) near the side ventilation port (21) may be bonded to the inner wall of the main ventilation tube (1) on the same side as the side ventilation port (21). Or, the side breathing port (24) may be located on the wall of the main ventilation tube (1) opposite to the side ventilation port (21). The two ends of the ventilation membrane tube (2) may be sealed and connected to the main ventilation port (11) and the side breathing port (24), respectively. The outer wall of the ventilation membrane tube (2) near the side breathing port (24) may be bonded to the inner wall of the main ventilation tube (1) on the same side as the side ventilation port (24). The inner walls on the same side are bonded together, and the outer wall of the ventilation membrane tube (2) is bonded to the inner wall of the main ventilation tube (1) between the side ventilation port (21) and the main ventilation port (11).
